(19) United States
(12) Patent Application Publication
(10) Pub. No.: US 2025/0204923 A1
(43) Pub. Date: Jun. 26, 2025

(54) LEFT ATRIAL APPENDAGE IMPLANT

(71) Applicant: BOSTON SCIENTIFIC SCIMED, INC., MAPLE GROVE, MN (US)

(72) Inventors: Patrick Willoughby, Shoreview, MN (US); Joshua Mark Inouye, Brooklyn Park, MN (US)

(73) Assignee: BOSTON SCIENTIFIC SCIMED, INC., MAPLE GROVE, MN (US)

(21) Appl. No.: 18/987,183

(22) Filed: Dec. 19, 2024

Related U.S. Application Data
(60) Provisional application No. 63/612,493, filed on Dec. 20, 2023.
(60) Provisional application No. 63/612,507, filed on Dec. 20, 2023.
(60) Provisional application No. 63/612,569, filed on Dec. 20, 2023.
(60) Provisional application No. 63/612,582, filed on Dec. 20, 2023.
(60) Provisional application No. 63/561,406, filed on Mar. 5, 2024.
(60) Provisional application No. 63/561,415, filed on Mar. 5, 2024.
(60) Provisional application No. 63/560,160, filed on Mar. 1, 2024.
(60) Provisional application No. 63/560,174, filed on Mar. 1, 2024.

(51) Int. Cl. A61B 17/12; A61B 17/00

(52) U.S. Cl. CPC A61B 17/12172; A61B 2017/00477; A61B 2017/00862; A61B 2017/12054

(57) ABSTRACT

An occlusive implant system includes a core wire having a lumen extending therethrough, an occlusive implant including an expandable framework releasably coupled to a distal end of the core wire, and an elongate strand extending along the core wire to the framework. The elongate strand extends into an interior of the framework. At least one piece of expandable foam may be configured to slide in a compressed configuration along the elongate strand into the interior of the framework. The at least one piece of expandable foam is configured to shift to an expanded configuration within the interior of the framework. The system may include a retaining structure holding at least one piece of expandable foam in the compressed configuration within the framework, wherein the retaining structure is engaged with the framework. The elongate strand may extend into the interior of the framework and be coupled to the retaining structure.

### CROSS-REFERENCE TO RELATED APPLICATIONS

This application claims the benefit of priority of U.S. Provisional Application No. 63/612,493 filed Dec. 20, 2023, 63/612,507, filed Dec. 20, 2023, 63/612,569, filed Dec. 20, 2023, 63/612,582, filed Dec. 20, 2023, 63/561,406, filed Mar. 5, 2024, 63/561,415, filed Mar. 5, 2024, 63/560,160, filed Mar. 1, 2024, and 63/560,174, filed Mar. 1, 2024, the entirety disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

In one example, an occlusive implant system may comprise a core wire having a lumen extending therethrough, an occlusive implant releasably coupled to a distal end of the core wire, the occlusive implant comprising an expandable framework configured to shift between a collapsed configuration and a deployed configuration, and an elongate strand extending along the core wire to the expandable framework. The elongate strand may extend into an interior of the expandable framework in the deployed configuration.

In addition or alternatively to any example described herein, in another example, the elongate strand extends within the lumen of the core wire to the expandable framework.

In addition or alternatively to any example described herein, in another example, a distal end of the elongate strand is secured to the expandable framework.

In addition or alternatively to any example described herein, in another example, the distal end of the elongate strand is secured to a proximal portion of the expandable framework.

In addition or alternatively to any example described herein, in another example, the distal end of the elongate strand is secured to a distal hub of the expandable framework.

In addition or alternatively to any example described herein, in another example, the elongate strand is configured to remain coupled to the expandable framework after the core wire has been decoupled from the expandable framework.

In addition or alternatively to any example described herein, in another example, the occlusive implant system may comprise an occlusive covering disposed on a proximal portion of the expandable framework.

In addition or alternatively to any example described herein, in another example, the occlusive implant system may comprise at least one piece of expandable foam configured to slide over the elongate strand into the interior of the expandable framework, the at least one piece of expandable foam being configured to remain within the interior of the expandable framework permanently.

In addition or alternatively to any example described herein, in another example, an occlusive implant system may comprise a core wire having a lumen extending therethrough, an occlusive implant releasably coupled to a distal end of the core wire, the occlusive implant comprising an expandable framework configured to shift between a collapsed configuration and a deployed configuration, an elongate strand extending along the core wire to the expandable framework, wherein the elongate strand extends into an interior of the expandable framework in the deployed configuration, and at least one piece of expandable foam configured to slide in a compressed configuration along the elongate strand into the interior of the expandable framework, wherein the at least one piece of expandable foam is configured to shift to an expanded configuration within the interior of the expandable framework.

In addition or alternatively to any example described herein, in another example, in the expanded configuration the at least one piece of expandable foam is configured to fill at least 50% of the interior of the expandable framework in the deployed configuration.

In addition or alternatively to any example described herein, in another example, a first portion of the elongate strand extends within the lumen of the core wire to the expandable framework.

In addition or alternatively to any example described herein, in another example, a second portion of the elongate strand extends alongside and external to the core wire to the expandable framework.

In addition or alternatively to any example described herein, in another example, a first piece of the at least one piece of expandable foam is configured to slide along the first portion of the elongate strand into the interior of the expandable framework and a second piece of the at least one piece of expandable foam is configured to slide along the second portion of the elongate strand into the interior of the expandable framework.

In addition or alternatively to any example described herein, in another example, the at least one piece of expandable foam is disposed outside of the expandable framework prior to shifting the expandable framework to the deployed configuration.

In addition or alternatively to any example described herein, in another example, an occlusive implant system may comprise a core wire having a lumen extending therethrough, an occlusive implant releasably coupled to a distal end of the core wire, the occlusive implant comprising an expandable framework configured to shift between a collapsed configuration and a deployed configuration, at least one piece of expandable foam disposed in a compressed configuration within an interior of the expandable framework in the collapsed configuration, a retaining structure holding the at least one piece of expandable foam in the compressed configuration, wherein the retaining structure is engaged with the expandable framework in a retaining configuration, and an elongate strand extending along the core wire to the expandable framework, wherein the elongate strand extends into the interior of the expandable framework and is coupled to the retaining structure.

In addition or alternatively to any example described herein, in another example, proximal translation of the elongate strand is configured to shift the retaining structure from the retaining configuration to a release configuration, thereby releasing the at least one piece of expandable foam within the interior of the expandable framework.

In addition or alternatively to any example described herein, in another example, at least a portion of the retaining structure is configured to be removed from the interior of the expandable framework after releasing the at least one piece of expandable foam within the interior of the expandable framework.

In addition or alternatively to any example described herein, in another example, the retaining structure comprises a plurality of elongate fingers fixedly secured to each other proximate a first end of the plurality of elongate fingers and biased apart from each other proximate a second end of the plurality of elongate fingers opposite the first end.

In addition or alternatively to any example described herein, in another example, the retaining structure comprises a coupling element configured to prevent the plurality of elongate fingers from moving apart from each other proximate the second end of the plurality of elongate fingers.

In addition or alternatively to any example described herein, in another example, proximal translation of the elongate strand is configured to disengage the coupling element and the plurality of elongate fingers from each other.

The above summary of some embodiments, aspects, and/or examples is not intended to describe each embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-2 are side views illustrating selected aspects of an occlusive implant system for occluding a left atrial appendage;

FIGS. 3-4 illustrate selected aspects of an occlusive implant for occluding a left atrial appendage;

FIG. 5 illustrates selected aspects of a prior art configuration of the occlusive implant system of FIGS. 1-2;

FIGS. 6-8 illustrate selected aspects of the occlusive implant system of FIGS. 1-2 including an elongate strand in accordance with the disclosure;

FIGS. 9-10 illustrate selected aspects of the occlusive implant system including an elongate strand in accordance with the disclosure;

FIGS. 11-12 illustrate selected aspects of the occlusive implant system including an elongate strand in accordance with the disclosure;

FIGS. 13-15 illustrate selected aspects of the occlusive implant system including a retaining structure disposed within the interior of the occlusive implant;

FIGS. 16-18 illustrate selected aspects of the occlusive implant system including a retaining structure disposed within the interior of the occlusive implant;

FIGS. 19-22 illustrate selected aspects of the retaining structure in accordance with the disclosure; and

FIGS. 23-25 illustrates selected aspects of the occlusive implant system including an elongate strand in accordance with the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s).

Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

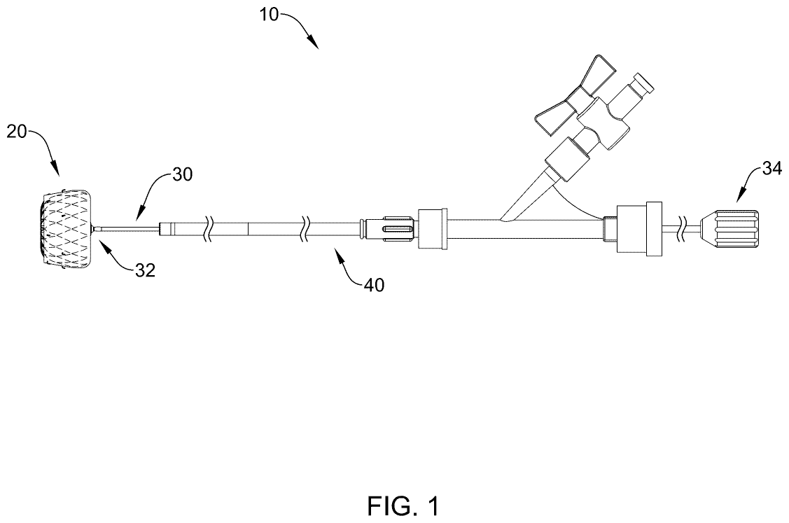

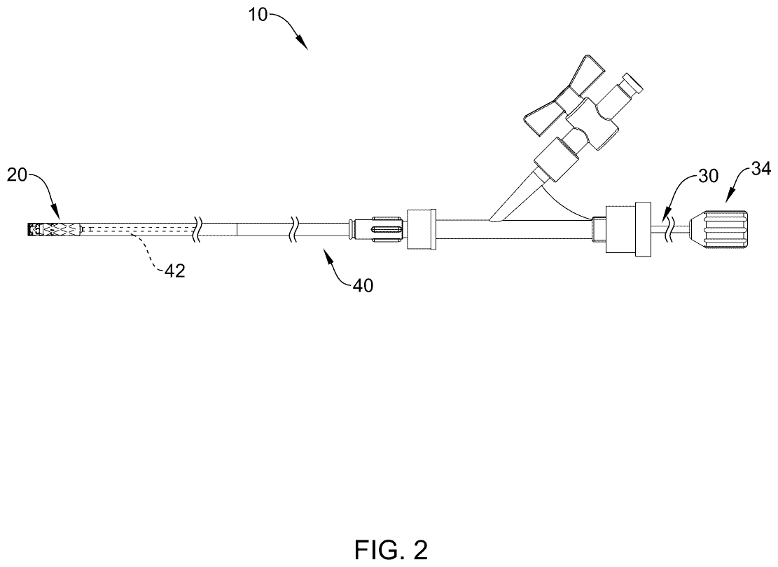

FIGS. 1-2 illustrate selected components and/or arrangements of an occlusive implant system 10 for occluding a left atrial appendage. It should be noted that in any given figure, some features of the occlusive implant system 10 may not be shown, or may be shown schematically, for simplicity. Additional details regarding some of the components of the occlusive implant system 10 may be illustrated in other figures in greater detail. The occlusive implant system 10 may be used to percutaneously deliver and/or deploy a variety of medical implants (e.g., a cardiovascular implant, an occlusive implant, a replacement heart valve implant, etc.) to one or more locations within the anatomy, including but not limited to, in some embodiments, the heart.

The occlusive implant system 10 may comprise a core wire 30. The occlusive implant system 10 may comprise an occlusive implant 20 releasably coupled to and/or disposed at a distal end 32 of the core wire 30. In at least some embodiments, the occlusive implant 20 may be configured to occlude the left atrial appendage. The left atrial appendage is attached to and in fluid communication with the left atrium of the patient's heart. The left atrial appendage may have a complex geometry and/or irregular surface area.

In some embodiments, the occlusive implant system 10 may include a delivery sheath 40 having a lumen 42 (e.g., FIG. 2) extending from a proximal opening to a distal opening. In some embodiments, the core wire 30 may be slidably disposed within the lumen 42 of the delivery sheath 40. The core wire 30 may have a proximal end 34 disposed proximal of the delivery sheath 40. In some embodiments, the proximal end 34 of the core wire 30 may include a knob and/or a handle configured to manipulate and/or move the core wire 30 and/or the occlusive implant 20. In some embodiments, the proximal end 34 of the core wire 30 may include a knob and/or a handle configured to manipulate and/or move the core wire 30 and/or the occlusive implant 20 relative to the delivery sheath 40. In some embodiments, the delivery sheath 40 may be sized and configured to deliver the occlusive implant 20 to the left atrial appendage.

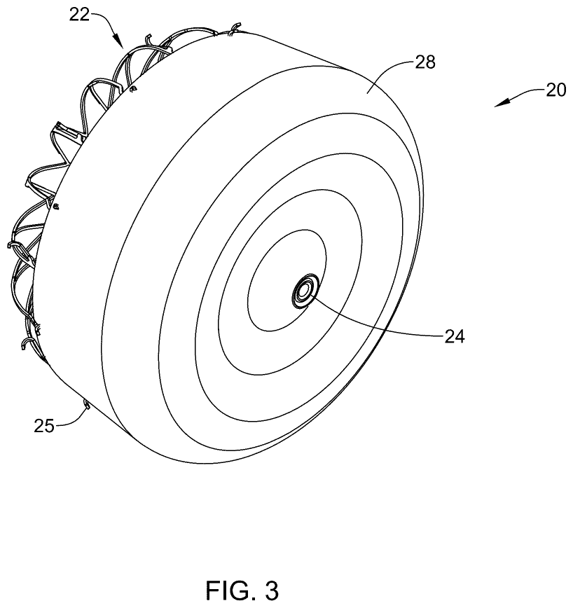

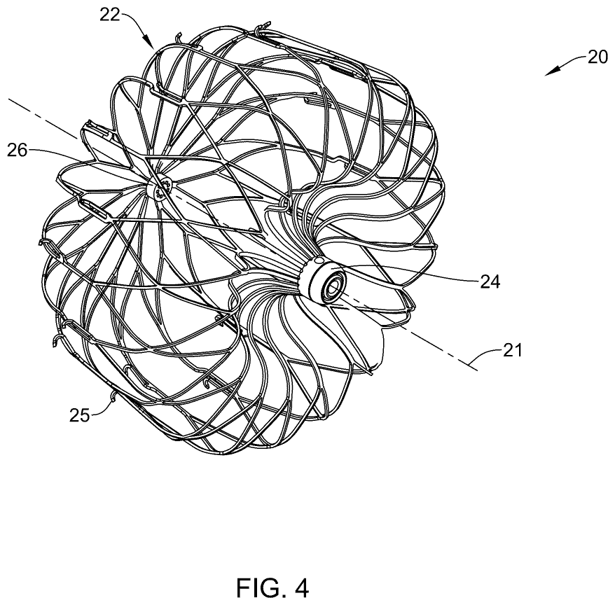

The occlusive implant 20 may include an expandable framework 22 (e.g., FIGS. 3-4) configured to shift between a collapsed configuration (e.g., FIG. 2) and a deployed configuration (e.g., FIG. 1). In some embodiments, the occlusive implant 20 may be disposed within the lumen 42 proximate the distal opening in the collapsed configuration. In some embodiments, the delivery sheath 40 may constrain the occlusive implant 20 and/or the expandable framework 22 in the collapsed configuration. In some embodiments, the occlusive implant 20 and/or the expandable framework 22 may be configured to shift between the collapsed configuration and the deployed configuration when the occlusive implant 20 is disposed distal of the distal opening of the lumen 42 and/or the delivery sheath 40, and/or when the occlusive implant 20 is unconstrained. In some embodiments, the occlusive implant 20 and/or the expandable framework 22 may be configured to shift between the collapsed configuration and the deployed configuration when the occlusive implant 20 is unconstrained by the delivery sheath 40. In at least some embodiments, the expandable framework 22 may be self-biased toward the deployed configuration.

In some embodiments, the core wire 30 may be slidably and/or rotatably disposed within the lumen 42 of the delivery sheath 40. In some embodiments, the proximal end 34 of the core wire 30 may extend proximally of a proximal end of the delivery sheath 40 and/or the proximal opening of the lumen 42 for manual manipulation by a clinician or practitioner. In some embodiments, the occlusive implant 20 may be removably attached, joined, secured, or otherwise connected to the distal end 32 of the core wire 30. The core wire 30 may be configured to and/or may be capable of axially translating the occlusive implant 20 relative to the delivery sheath 40. The delivery sheath 40 and/or the core wire 30 may have a selected level of axial stiffness and/or pushability characteristics while also having a selected level of flexibility to permit navigation through the patient's vasculature.

Some suitable, but non-limiting, examples of materials for the occlusive implant system 10, the core wire 30, the delivery sheath 40, and/or the occlusive implant 20, etc. are discussed below.

Turning now to FIGS. 3 and 4, the occlusive implant 20 may comprise an expandable framework 22 configured to shift along a longitudinal axis 21 (e.g., FIG. 4) between the collapsed configuration and the deployed configuration. In the collapsed configuration, the expandable framework 22 may be axially elongated and/or radially compressed. In the deployed configuration, the expandable framework 22 may be axially shortened and/or radially expanded. The expandable framework 22 may comprise a plurality of interconnected struts defining a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements. In some embodiments, the plurality of interconnected struts may converge, join, and/or connect at intersections or nodes.

The plurality of interconnected struts may be formed and/or cut from a tubular member. In some embodiments, the plurality of interconnected struts may be integrally formed and/or cut from a unitary member. In some embodiments, the plurality of interconnected struts may be integrally formed and/or cut from a unitary tubular member and subsequently formed and/or heat set to a desired shape in the deployed configuration. In some embodiments, the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the plurality of interconnected struts include laser cutting, machining, punching, stamping, electro discharge machining (EDM), chemical dissolution, etc. Other means and/or methods are also contemplated.

In some embodiments, the expandable framework 22 may be compliant and substantially conform to and/or be in sealing engagement with the shape and/or geometry of a wall of the left atrial appendage in the deployed configuration. In some embodiments, the occlusive implant 20 may expand to a size, extent, or shape less than or different from a maximum unconstrained extent, as determined by the surrounding tissue and/or wall of the left atrial appendage. In some embodiments, reducing a thickness of various elements of the expandable framework 22 may increase the flexibility and compliance of the expandable framework 22 and/or the occlusive implant 20, thereby permitting the expandable framework 22 and/or the occlusive implant 20 to conform to the tissue around it, rather than forcing the tissue to conform to the expandable framework 22 and/or the occlusive implant 20. In some embodiments, the expandable framework 22 and/or the occlusive implant 20 may be stronger and/or less compliant, and thus the expandable framework 22 and/or the occlusive implant 20 may force the tissue of the left atrial appendage to conform to the expandable framework 22 and/or the occlusive implant 20 in the deployed configuration. Other configurations are also contemplated.

In some embodiments, the occlusive implant 20 and/or the expandable framework 22 may comprise a plurality of anchoring elements 25. In some embodiments, the plurality of anchoring elements 25 may extend radially outward from the expandable framework 22 in the deployed configuration. In at least some embodiments, the plurality of anchoring elements 25 may be configured to engage with tissue and/or may be configured to secure the occlusive implant 20 and/or the expandable framework 22 to tissue at a target site (e.g., the left atrial appendage, etc.). In some embodiments, the plurality of anchoring elements 25 may be configured to prevent dislodgement and/or ejection of the occlusive implant 20 from the target site.

In some embodiments, the occlusive implant 20 and/or the expandable framework 22 may include a proximal hub 24 and a distal hub 26. The longitudinal axis 21 of the expandable framework 22 may extend from the proximal hub 24 to the distal hub 26. In at least some embodiments, the proximal hub 24 and/or the distal hub 26 may be centered on and/or coaxial with the longitudinal axis 21. The plurality of interconnected struts may be joined together at and/or fixedly attached to the proximal hub 24 and/or the distal hub 26. In some embodiments, the proximal hub 24 and/or the distal hub 26 may be fixedly attached to the expandable framework 22 and/or the plurality of interconnected struts, such as by welding, adhesive bonding, brazing, soldering, etc. The proximal hub 24 may be configured to releasably connect, couple, and/or attach the occlusive implant 20 and/or the expandable framework 22 to the distal end 32 of the core wire 30 (e.g., FIGS. 1-2). In some embodiments, the proximal hub 24 may include internal threads configured to rotatably and/or threadably engage external threads formed on and/or at the distal end 32 of the core wire 30. Other configurations for releasably securing the occlusive implant 20 to the core wire 30 are also contemplated.

In some embodiments, the occlusive implant 20 may optionally include an occlusive covering 28 connected to, disposed on, disposed over, disposed about, and/or disposed radially outward of a proximal portion of the expandable framework 22 and/or the plurality of interconnected struts. In some embodiments, the occlusive covering 28 may be attached to the proximal hub 24 and/or may be attached to the expandable framework at the proximal hub 24. In some embodiments, the occlusive covering 28 may extend radially outward from and/or may extend distally from the proximal hub 24. In some embodiments, the occlusive covering 28 may be attached and/or secured to the expandable framework 22 at a plurality of discrete locations. In some embodiments, one or more of the plurality of anchoring elements 25 may extend through the occlusive covering 28. In some embodiments, the one or more of the plurality of anchoring elements 25 extending through the occlusive covering 28 may attach and/or secure the occlusive covering 28 to the expandable framework 22.

In some embodiments, the occlusive covering 28 may include a membrane, a fabric, a mesh, a tissue element, or another suitable construction. In some embodiments, the occlusive covering 28 may be porous. In some embodiments, the occlusive covering 28 may be non-porous. In some embodiments, the occlusive covering 28 may be permeable or impermeable to blood and/or other fluids, such as water. In some embodiments, the occlusive covering 28 may be designed, sized, and/or configured to prevent thrombus and/or embolic material from passing out of the left atrial appendage into the left atrium and/or the patient's bloodstream. In some embodiments, the occlusive covering 28 (e.g., the membrane, the fabric, or the tissue element, etc.) promotes endothelization after implantation, thereby effectively and/or permanently removing the target site (e.g., the left atrial appendage, etc.) from the patient's circulatory system. Some suitable, but non-limiting, examples of materials for the occlusive covering 28 are discussed below.

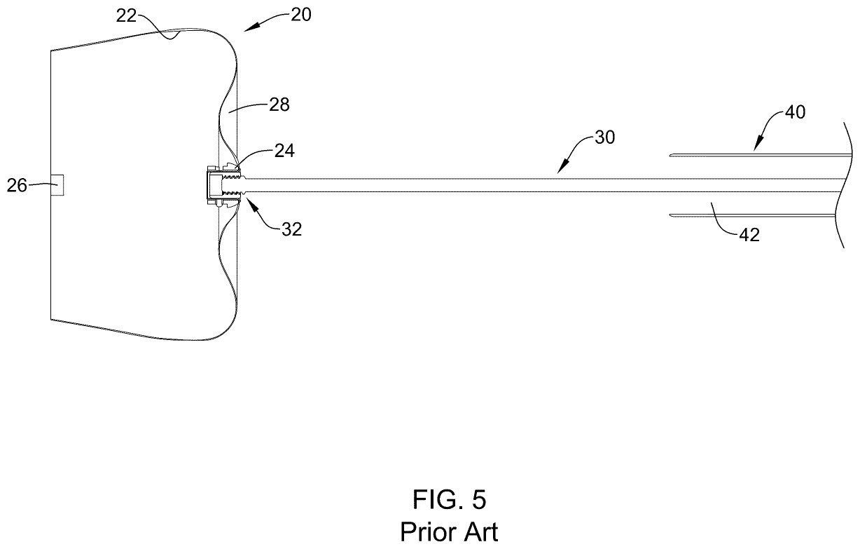

FIG. 5 is a partial cross-sectional view illustrating selected aspects of a prior art configuration of the core wire 30 coupled to the occlusive implant 20. As shown, the core wire 30 is a solid shaft with external threads formed at the distal end 32 of the core wire 30. The external threads engage with internal threads formed in the proximal hub 24 of the occlusive implant 20. The core wire is coupled only to the proximal hub 24. In some cases, the core wire 30 may be disposed within a hollow sheath (not shown). The core wire 30 is slidably disposed within the lumen 42 of the delivery sheath 40. In cases where the core wire 30 is disposed within a hollow sheath, the hollow sheath is disposed within the lumen 42 of the delivery sheath 40.

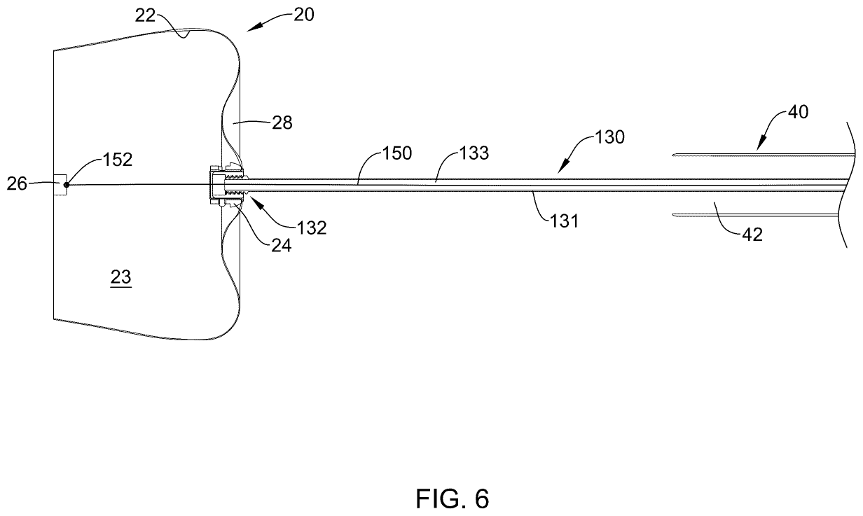

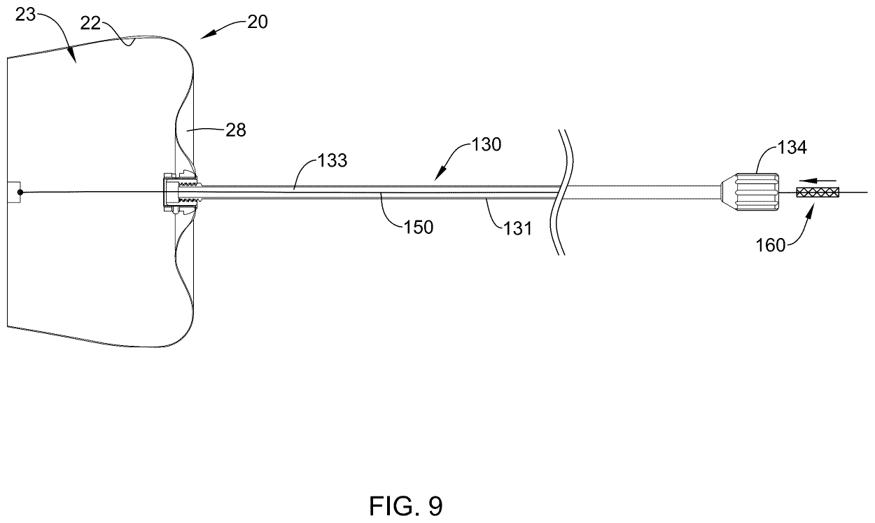

FIG. 6 is a partial cross-sectional view illustrating selected aspects of a core wire 130 according to the disclosure for use in the occlusive implant system 10. In addition or alternatively to other configurations disclosed herein, the occlusive implant system 10 may comprise the core wire 130 having a lumen 133 extending therethrough. The core wire 130 may be releasably coupled to the occlusive implant 20 at a distal end 132 of the core wire 130. In at least some embodiments, the distal end 132 of the core wire 130 may include external threads formed on and/or at the distal end 132 of the core wire 130, wherein the external threads are configured to rotatably and/or threadably engage the internal threads of the proximal hub 24. Other configurations are also contemplated. In some embodiments, the core wire 130 may comprise a wall 131 defining the lumen 133. In some embodiments, the wall 131 may extend from a proximal end 134 (e.g., FIGS. 9-12) to the distal end 132 of the core wire 130. In some embodiments, the lumen 133 may extend from the proximal end 134 (e.g., FIGS. 9-12) to the distal end 132 of the core wire 130.

In some embodiments, the core wire 130 may be formed as a tubular member. In some embodiments, the core wire 130 and/or the wall 131 may comprise a single-layered tube. In some embodiments, the core wire 130 and/or the wall 131 may comprise a multi-layered tube. In some embodiments, the core wire 130 and/or the wall 131 may comprise an outer jacket and a tubular coil disposed therein and/or embedded within the outer jacket. In some embodiments, the core wire 130 and/or the wall 131 may comprise an outer jacket and a tubular braid disposed therein and/or embedded within the outer jacket. Other configurations are also contemplated. In at least some embodiments, the core wire 130 and/or the wall 131 may be formed from a polymeric material. In some embodiments, the core wire 130 and/or the wall 131 may be formed from a metallic material. In some embodiments, the core wire 130 and/or the wall 131 may be formed from a combination of metallic and polymeric materials.

In some embodiments, the occlusive implant system 10 may comprise an elongate strand 150 extending along the core wire 130 to the occlusive implant 20 and/or the expandable framework 22. In some embodiments, the elongate strand 150 may comprise a suture, a filament, a wire, a thread, etc. In some embodiments, the elongate strand 150 may have a length that is fixed. In at least some embodiments, the length of the elongate strand 150 may be greater than a length of the core wire 130. In some embodiments, the elongate strand 150 may extend proximal of the proximal end 134 (e.g., FIGS. 9-12) of the core wire 130. In some embodiments, the elongate strand 150 may extend distal of the distal end 132 of the core wire 130. In some embodiments, the elongate strand 150 may extend proximal of the proximal end 134 (e.g., FIGS. 9-12) of the core wire 130 and distal of the distal end 132 of the core wire 130. In some embodiments, the core wire 130 may be rotatable relative to the elongate strand 150.

In some embodiments, the length of the elongate strand 150 may not change under load. In at least some embodiments, the elongate strand 150 may have minimal stretch or elongation. For example, as an axial force is applied to the elongate strand 150, the elongate strand 150 may resist and/or avoid stretching or elongating. In some embodiments, the elongate strand 150 may be axially inelastic. Alternatively, in some embodiments, the elongate strand 150 may be axially elastic and/or may be capable of elongating or stretching by a predetermined amount. Other configurations are also contemplated.

In some embodiments, the elongate strand 150 may extend alongside the core wire 130 to the occlusive implant 20 and/or the expandable framework 22. In some embodiments, the elongate strand 150 may extend within the lumen 133 of the core wire 130 to the occlusive implant 20 and/or the expandable framework 22. In some embodiments, the elongate strand 150 may extend alongside the core wire 130 for a first portion of its length and within the lumen 133 of the core wire 130 for a second portion of its length. Other configurations are also contemplated.

In at least some embodiments, the elongate strand 150 may extend into an interior 23 of the occlusive implant 20 and/or the expandable framework 22 in the deployed configuration. In some embodiments, a distal end 152 of the elongate strand 150 may be secured to, coupled to, and/or attached to the expandable framework 22 and/or the occlusive implant 20. In some embodiments, the distal end 152 of the elongate strand 150 may be fixedly attached to the expandable framework 22 and/or the occlusive implant 20. In some embodiments, the distal end 152 of the elongate strand 150 may be secured to, coupled to, and/or attached to the distal hub 26 of the expandable framework 22 and/or the occlusive implant 20, as seen in FIG. 6. In some embodiments, the distal end 152 of the elongate strand 150 may be fixedly attached to the distal hub 26 of the expandable framework 22 and/or the occlusive implant 20.

In some embodiments, the elongate strand 150 may comprise and/or include a loop. In some embodiments, a distal extent of the loop of the elongate strand 150 may be secured to, coupled to, and/or attached to the expandable framework 22 and/or the occlusive implant 20.

In some embodiments, the elongate strand 150 may be configured to manipulate the distal hub 26 and/or a distal portion of the expandable framework 22. In some embodiments, the elongate strand 150 may be configured to pull the distal hub 26 of the expandable framework 22 toward the proximal hub 24 of the expandable framework 22 and/or toward the distal end 132 of the core wire 130. In some embodiments, pulling on the elongate strand 150 may cause the expandable framework 22 to shift toward the deployed configuration and/or adjust the size of the expandable framework 22 during deployment.

In some embodiments, holding the core wire 130 fixed and pulling on the elongate strand 150 after the expandable framework 22 has been shifted toward and/or to the deployed configuration may urge the expandable framework 22 radially outward and/or may increase the radially outward force exerted by the expandable framework 22 against the target site (e.g., the left atrial appendage). In some embodiments, holding the core wire 130 fixed and pulling on the elongate strand 150 after the expandable framework 22 has been shifted toward and/or to the deployed configuration may cause the plurality of anchoring elements 25 to engage with and/or to increase their engagement with tissue at the target site (e.g., the left atrial appendage).

In some embodiments, tension applied to the elongate strand 150 after the expandable framework 22 has been shifted toward and/or to the deployed configuration may make the occlusive implant 20 and/or the expandable framework 22 more rigid and/or more maneuverable with the core wire 130.

In some embodiments, tension applied to the elongate strand 150 during deployment of the expandable framework 22 and/or after the expandable framework 22 has been shifted toward and/or to the deployed configuration may permit the occlusive implant 20 and/or the expandable framework 22 to be tilted and/or angled relative to a longitudinal axis of the core wire 130, such as when positioning or repositioning the occlusive implant 20 within the left atrial appendage.

In some embodiments, a tug test may be performed after the expandable framework 22 has been shifted toward and/or to the deployed configuration to verify implant stability and/or to verify that the occlusive implant 20 is secured to tissue at the target site (e.g., the left atrial appendage). In some embodiments, the tug test may be performed by pulling on the core wire 130, which may apply a proximal force to the proximal hub 24. In some situations, the tug test may cause the expandable framework 22 to partially revert towards the collapsed configuration thereby reducing the radially outward force exerted by the expandable framework 22 and/or disengaging the plurality of anchoring elements 25 from tissue at the target site (e.g., the left atrial appendage). Pulling proximally on the elongate strand 150 and/or the distal hub 26 during the tug test may maintain and/or increase the radially outward force exerted by the expandable framework 22 and/or may maintain and/or increase engagement of the plurality of anchoring elements 25 with tissue at the target site (e.g., the left atrial appendage).

In some embodiments, the elongate strand 150 may be configured to remain coupled to the expandable framework 22 after the core wire 130 has been decoupled from the expandable framework 22. For example, in configurations where the elongate strand 150 extends within the lumen 133 of the core wire 130, the core wire 130 may be decoupled from the proximal hub 24 and/or the expandable framework 22 to release tension and/or biasing force built up within the core wire 130 during placement of the occlusive implant 20 to ensure that the occlusive implant 20 remains in the desired placement. If a practitioner then determines that the occlusive implant 20 needs to be repositioned, the elongate strand 150 may act as a guide for recoupling the core wire 130 to the proximal hub 24 and/or the expandable framework 22. Other configurations and/or uses for the elongate strand 150 when secured to the distal hub 26 of the expandable framework 22 are also contemplated.

After satisfactory deployment of the occlusive implant 20, the elongate strand 150 may be decoupled from the expandable framework 22, severed along its length, and/or removed from the interior 23 of the occlusive implant 20. In some embodiments, the elongate strand 150 may be decoupled from the expandable framework 22, severed along its length, and/or removed from the interior 23 of the occlusive implant 20 prior to decoupling the core wire 130 from the proximal hub 24 and/or the expandable framework 22. In some embodiments, the elongate strand 150 may be decoupled from the expandable framework 22, severed along its length, and/or removed from the interior 23 of the occlusive implant 20 after decoupling the core wire 130 from the proximal hub 24 and/or the expandable framework 22. In some embodiments, the elongate strand 150 may be decoupled from the expandable framework 22, severed along its length, and/or removed from the interior 23 of the occlusive implant 20 while and/or simultaneously to decoupling the core wire 130 from the proximal hub 24 and/or the expandable framework 22.

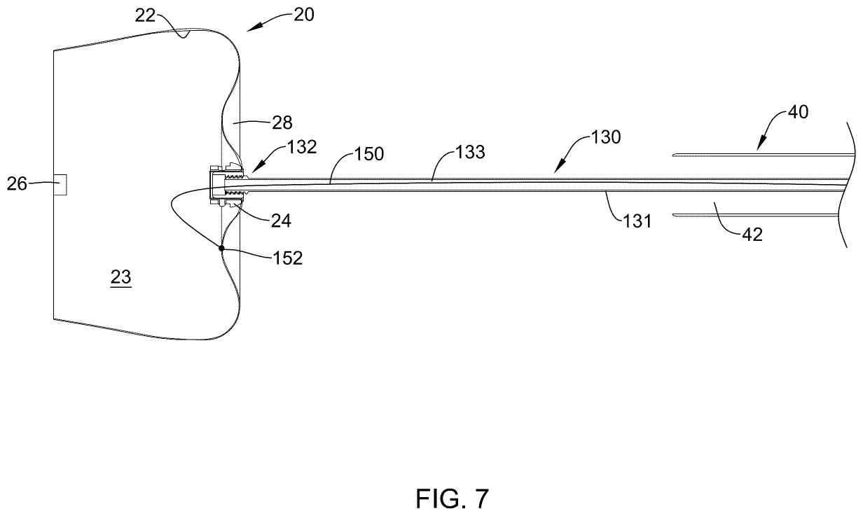

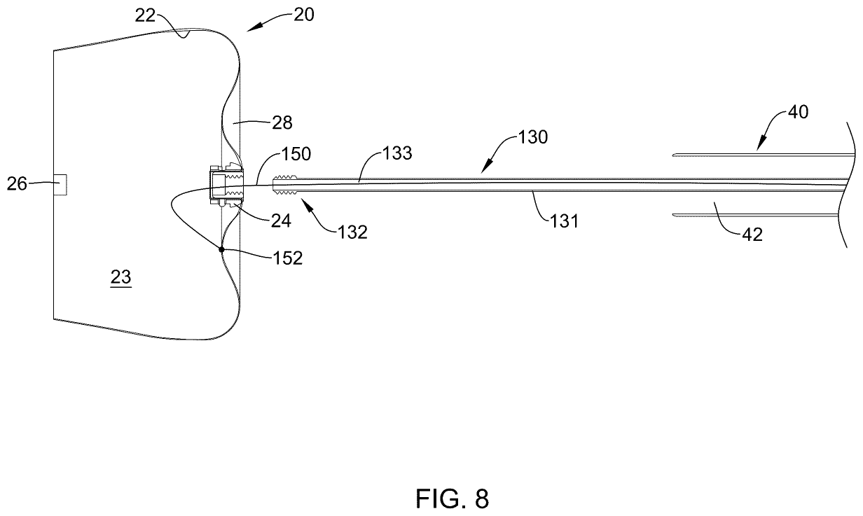

In some embodiments, the distal end 152 of the elongate strand 150 may be secured to, coupled to, and/or attached to a proximal portion of the expandable framework 22 and/or the occlusive implant 20, as seen in FIG. 7. In some embodiments, the distal end 152 of the elongate strand 150 may be fixedly attached to the proximal portion of the expandable framework 22 and/or the occlusive implant 20. In some embodiments, the elongate strand 150 may be configured to remain coupled to the expandable framework 22 after the core wire 130 has been decoupled from the expandable framework 22, as seen in FIG. 8. For example, in configurations where the elongate strand 150 extends within the lumen 133 of the core wire 130, the core wire 130 may be decoupled from the proximal hub 24 and/or the expandable framework 22 to release tension and/or biasing force built up within the core wire 130 during placement of the occlusive implant 20 to ensure that the occlusive implant 20 remains in the desired placement. If a practitioner then determines that the occlusive implant 20 needs to be repositioned, the elongate strand 150 may function as a guide for recoupling the core wire 130 to the proximal hub 24 and/or the expandable framework 22.

In some embodiments, tension applied to the elongate strand 150 during deployment of the expandable framework 22 and/or after the expandable framework 22 has been shifted toward and/or to the deployed configuration may apply a side load to the occlusive implant 20 and/or the expandable framework 22. In some embodiments, tension applied to the elongate strand 150 during deployment of the expandable framework 22 and/or after the expandable framework 22 has been shifted toward and/or to the deployed configuration may permit the occlusive implant 20 and/or the expandable framework 22 to be steered, tilted, and/or angled relative to a longitudinal axis of the core wire 130, such as when positioning or repositioning the occlusive implant 20 within the left atrial appendage. While such steering, tilting, and/or angling may generally be limited to a single direction, the proximal end 134 of the core wire 130 may be rotated relative to the delivery sheath to effect steering of the occlusive implant 20 and/or the expandable framework 22. Other configurations and/or uses for the elongate strand 150 when secured to the proximal portion of the expandable framework 22 are also contemplated.

After satisfactory deployment of the occlusive implant 20, the elongate strand 150 may be decoupled from the expandable framework 22, severed along its length, and/or removed from the interior 23 of the occlusive implant 20. In some embodiments, the elongate strand 150 may be decoupled from the expandable framework 22, severed along its length, and/or removed from the interior 23 of the occlusive implant 20 prior to decoupling the core wire 130 from the proximal hub 24 and/or the expandable framework 22. In some embodiments, the elongate strand 150 may be decoupled from the expandable framework 22, severed along its length, and/or removed from the interior 23 of the occlusive implant 20 after decoupling the core wire 130 from the proximal hub 24 and/or the expandable framework 22. In some embodiments, the elongate strand 150 may be decoupled from the expandable framework 22, severed along its length, and/or removed from the interior 23 of the occlusive implant 20 while and/or simultaneously to decoupling the core wire 130 from the proximal hub 24 and/or the expandable framework 22.

In some embodiments, the occlusive implant system 10 may comprise at least one piece of expandable foam 160 configured to slide along and/or over the elongate strand 150 into the interior 23 of the expandable framework 22, as seen in FIGS. 9-12. In some embodiments, the at least one piece of expandable foam 160 may be configured to slide in a compressed configuration along and/or over the elongate strand 150 into the interior 23 of the expandable framework 22. The at least one piece of expandable foam 160 may be configured to shift to an expanded configuration within the interior 23 of the expandable framework 22. In at least some embodiments, the at least one piece of expandable foam 160 may be disposed outside of the expandable framework 22 prior to shifting the occlusive implant 20 and/or the expandable framework 22 to the deployed configuration.

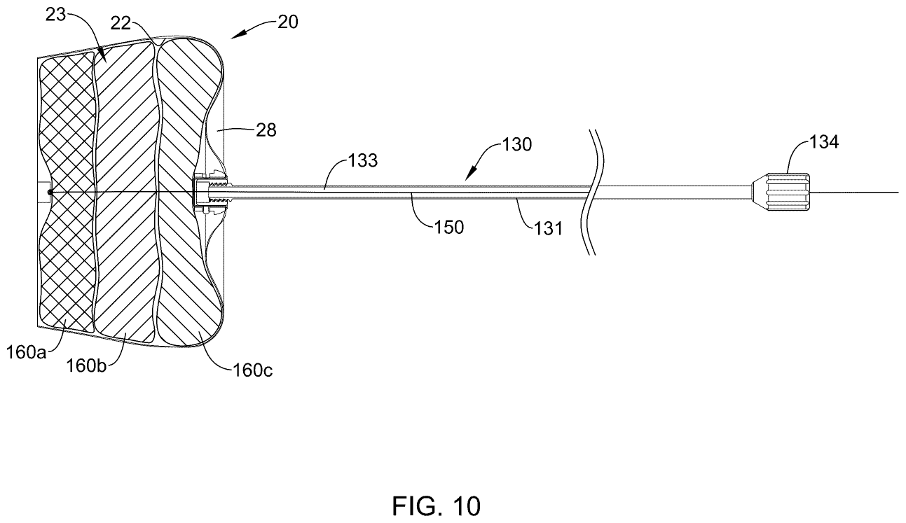

In some embodiments, the at least one piece of expandable foam 160 may comprise a plurality of pieces of expandable foam. In some embodiments, the at least one piece of expandable foam 160 and/or the plurality of pieces of expandable foam may be delivered to and/or advanced into the interior 23 of the expandable framework 22 sequentially. In one example, the at least one piece of expandable foam 160 and/or the plurality of pieces of expandable foam may be delivered to and/or advanced into the interior 23 of the expandable framework 22 sequentially as first, second, and third pieces of expandable foam, identified as reference numbers 160a-c in FIG. 10. Other configurations and/or examples, including fewer or greater pieces of expandable foam, are also contemplated.

In some embodiments, in the expanded configuration, the at least one piece of expandable foam 160 and/or the plurality of pieces of expandable foam may be configured to fill at least 50% of the interior 23 of the expandable framework 22 in the deployed configuration. In some embodiments, in the expanded configuration, the at least one piece of expandable foam 160 and/or the plurality of pieces of expandable foam may be configured to fill at least 60% of the interior 23 of the expandable framework 22 in the deployed configuration. In some embodiments, in the expanded configuration, the at least one piece of expandable foam 160 and/or the plurality of pieces of expandable foam may be configured to fill at least 75% of the interior 23 of the expandable framework 22 in the deployed configuration. In some embodiments, in the expanded configuration, the at least one piece of expandable foam 160 and/or the plurality of pieces of expandable foam may be configured to fill at least 80% of the interior 23 of the expandable framework 22 in the deployed configuration. In some embodiments, in the expanded configuration, the at least one piece of expandable foam 160 and/or the plurality of pieces of expandable foam may be configured to fill at least 85% of the interior 23 of the expandable framework 22 in the deployed configuration. In some embodiments, in the expanded configuration, the at least one piece of expandable foam 160 and/or the plurality of pieces of expandable foam may be configured to fill at least 90% of the interior 23 of the expandable framework 22 in the deployed configuration. In some embodiments, in the expanded configuration, the at least one piece of expandable foam 160 and/or the plurality of pieces of expandable foam may be configured to fill at least 95% of the interior 23 of the expandable framework 22 in the deployed configuration.

In some embodiments, the at least one piece of expandable foam 160 and/or the plurality of pieces of expandable foam may be configured to remain within the interior 23 of the expandable framework 22 permanently (e.g., the at least one piece of expandable foam 160 and/or the plurality of pieces of expandable foam is never removed from the interior 23 of the expandable framework 22 by the practitioner). In some embodiments, the at least one piece of expandable foam 160 and/or the plurality of pieces of expandable foam may be configured to be biodegradable over time. In some embodiments, the at least one piece of expandable foam 160 and/or the plurality of pieces of expandable foam may be configured to be biodegradable over at least 30 days' time. In some embodiments, the at least one piece of expandable foam 160 and/or the plurality of pieces of expandable foam may be configured to be biodegradable over at least 60 days' time. In some embodiments, the at least one piece of expandable foam 160 and/or the plurality of pieces of expandable foam may be configured to be biodegradable over at least 90 days' time. In some embodiments, the at least one piece of expandable foam 160 and/or the plurality of pieces of expandable foam may be configured to be biodegradable over at least 180 days' time.

In some embodiments, the at least one piece of expandable foam 160 and/or the plurality of pieces of expandable foam may be configured to be biodegradable over at least 365 days' time. Other configurations are also contemplated.

In some embodiments, the at least one piece of expandable foam 160 may be configured to prevent thrombus formation (e.g., within the left atrial appendage). In some embodiments, the at least one piece of expandable foam 160 may include anti-thrombus medicament(s). In some embodiments, the at least one piece of expandable foam 160 may be configured to absorb blood and/or bodily fluid(s). In some embodiments, the at least one piece of expandable foam 160 may be configured to trap thrombus. In some embodiments, the at least one piece of expandable foam 160 may be configured to promote tissue ingrowth and/or endothelization. Other configurations are also contemplated.

In at least some embodiments, the at least one piece of expandable foam 160 and/or the plurality of pieces of expandable foam may comprise and/or may be formed from a shape memory polymer and/or a shape memory foam. The shape memory polymer and/or the shape memory foam may have multiple geometric and/or mechanical properties when exposed to temperature, moisture, and/or chemical environments, and/or changes therein.

In some embodiments, the shape memory polymer and/or the shape memory foam may have a collapsibility ratio that is high. The collapsibility ratio is a ratio between an expanded size and a collapsed size. In some examples, the collapsibility ratio of the shape memory polymer and/or the shape memory foam may be at least 5 times, at least 7 times, at least 8 times, at least 9 times, at least 10 times, at least 12 times, or more. In one example, a piece of expandable foam may have an outer diameter of about 32 millimeters in the expanded configuration and about 3 millimeters in the compressed configuration, producing a collapsibility ratio of at least 10 times (e.g., at least 10:1). Other configurations are also contemplated. In at least some embodiments, the at least one piece of expandable foam 160 and/or the plurality of pieces of expandable foam may be configured as open celled foam.

In some embodiments, the at least one piece of expandable foam 160 and/or the plurality of pieces of expandable foam may be unconstrained by outside forces and/or structure(s) in the collapsed configuration. In some embodiments, the at least one piece of expandable foam 160 and/or the plurality of pieces of expandable foam may be self-maintained in the collapsed configuration by shape memory properties. Other configurations are also contemplated.

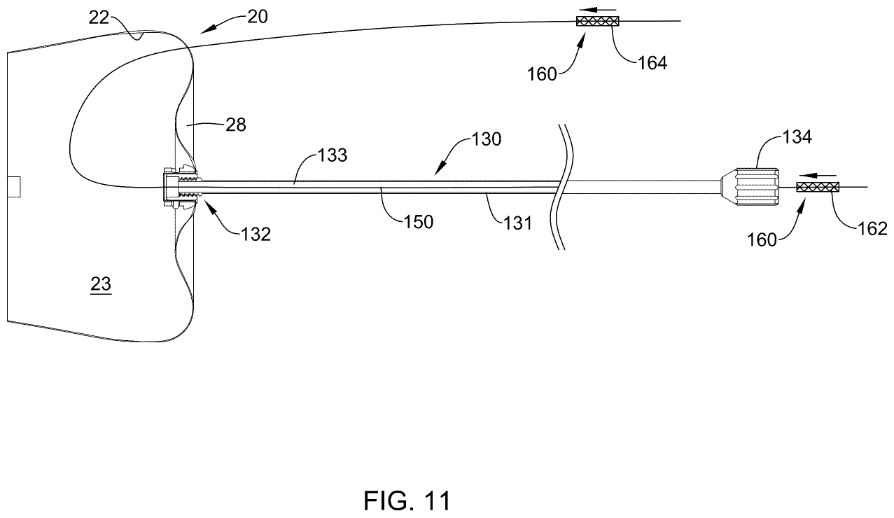

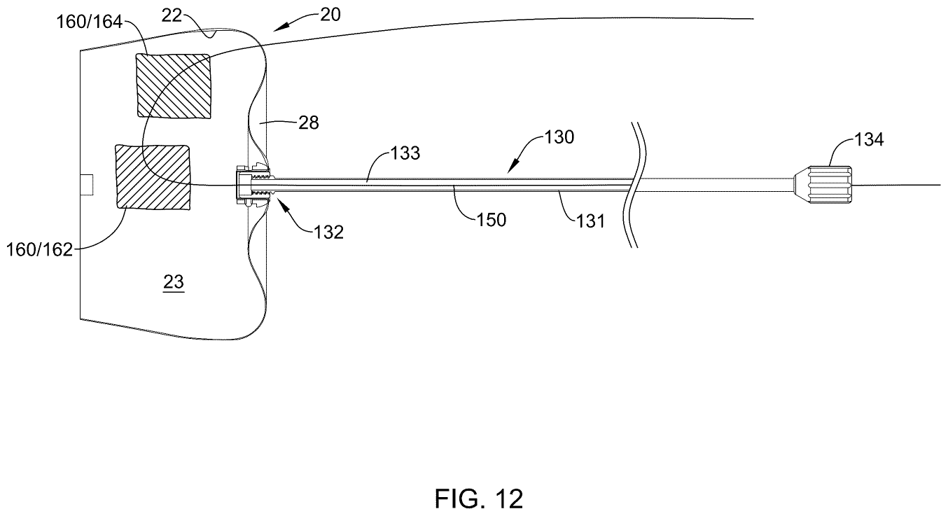

In some embodiments, a first portion of the elongate strand 150 may extend within the lumen 133 of the core wire 130 to the expandable framework 22 and/or into the interior 23 of the expandable framework 22 in the deployed configuration. In some embodiments, a second portion of the elongate strand 150 may extend alongside and/or external to the core wire 130 to the expandable framework 22 and/or into the interior 23 of the expandable framework 22 in the deployed configuration, as seen in FIGS. 11-12. In some embodiments, the first portion of the elongate strand 150 may extend within the lumen 133 of the core wire 130 and the second portion of the elongate strand 150 may extend alongside and/or external to the core wire 130 and inside of the lumen 42 of the delivery sheath 40. Other configurations are also contemplated.

In some embodiments, the first portion of the elongate strand 150 and the second portion of the elongate strand 150 may meet and/or join within the interior 23 of the expandable framework 22 in the deployed configuration. In some embodiments, the elongate strand 150 may be a single unitary and/or monolithic structure. In one example, the elongate strand 150 may comprise the first portion and the second portion in a single unitary and/or monolithic structure.

In some embodiments, the elongate strand 150 may comprise a first end and a second end opposite the first end. In some embodiments, the first end may be disposed at a proximal end of the first portion. In some embodiments, the second end may be disposed at a proximal end of the second portion. In at least some embodiments, the first end and the second end of the elongate strand 150 may be disposed proximal of the occlusive implant 20, proximal of the proximal end 134 of the core wire 130, and/or outside of the patient. Other configurations are also contemplated.

In some embodiments, a first piece 162 of the at least one piece of expandable foam 160 may be configured to slide along and/or over the first portion of the elongate strand 150 into the interior 23 of the expandable framework 22. In some embodiments, the first piece 162 of the at least one piece of expandable foam 160 may be configured to slide within the lumen 133 of the core wire 130 into the interior 23 of the expandable framework 22.

In some embodiments, a second piece 164 of the at least one piece of expandable foam 160 may be configured to slide along and/or over the second portion of the elongate strand 150 into the interior 23 of the expandable framework 22. In some embodiments, the second piece 164 of the at least one piece of expandable foam 160 may be configured to slide alongside and/or external to the core wire 130 into the interior 23 of the expandable framework 22.

In some embodiments, additional pieces of the at least one piece of expandable foam 160 may be configured to slide along and/or over the first portion of the elongate strand 150 and/or the second portion of the elongate strand 150 into the interior 23 of the expandable framework 22. In some embodiments, a sufficient number of pieces of the at least one piece of expandable foam 160 may be configured to slide along and/or over the first portion of the elongate strand 150 and/or the second portion of the elongate strand 150 into the interior 23 of the expandable framework 22 such that upon all pieces of the at least one piece of expandable foam 160 shifting to the expanded configuration, the interior 23 of the expandable framework 22 may be substantially filled (e.g., at least 50% filled, at least 60% filled, at least 75% filled, at least 80% filled, at least 85% filled, at least 90% filled, at least 95% filled, etc.) with expandable foam.

In some alternative embodiments, one or more medical devices may be advanced along and/or over the elongate strand 150 to the expandable framework 22. In some embodiments, one or more medical devices may be advanced along and/or over the elongate strand 150 into the interior 23 of the expandable framework 22. In some embodiments, the one or more medical devices may be used to perform a secondary procedure within the interior 23 of the expandable framework 22. In some embodiments, the one or more medical devices may be left within the interior 23 of the expandable framework 22 permanently. Other configurations are also contemplated. In some embodiments, the one or more medical devices may include occlusive devices or elements, sensors, embolic protection devices, heart pacing devices, imaging devices, therapy devices, or other suitable medical devices, including but not limited to, combinations thereof.

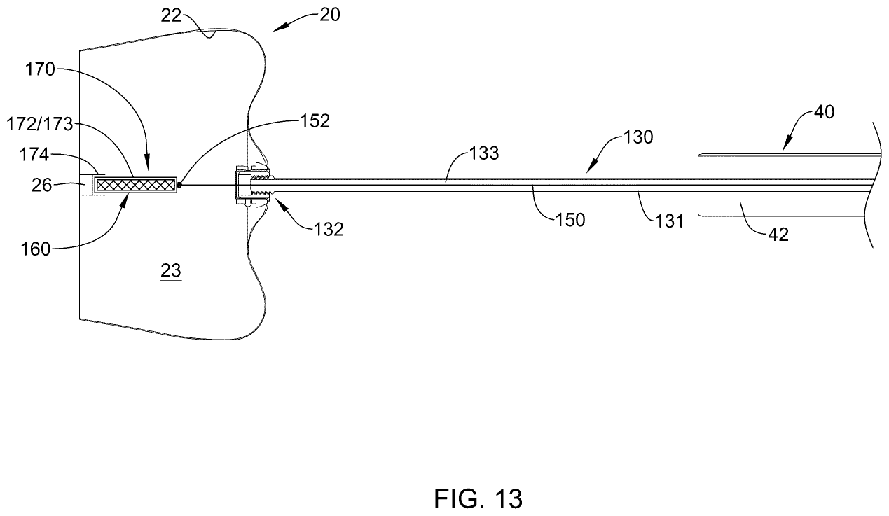

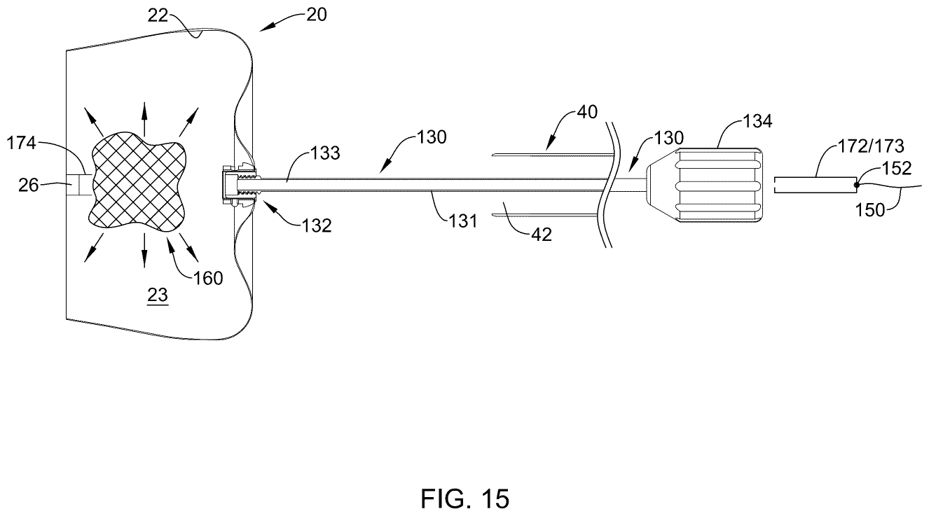

In some embodiments, the occlusive implant system 10 may comprise the core wire 130 having the lumen 133 extending therethrough, the occlusive implant 20 releasably coupled to the distal end 132 of the core wire 130, at least one piece of expandable foam 160 disposed in a compressed configuration within the interior 23 of the expandable framework 22 in the collapsed configuration (e.g., FIG. 2), a retaining structure 170 holding the at least one piece of expandable foam 160 in the compressed configuration, wherein the retaining structure 170 is engaged with the expandable framework 22 in a retaining configuration, and the elongate strand 150 extending along the core wire 130 to the expandable framework 22 and/or into the interior 23 of the expandable framework 22, as seen in FIGS. 13-16. In at least some embodiments, the distal end 152 of the elongate strand 150 may be secured to, coupled to, and/or attached to the retaining structure 170. In FIGS. 13 and 15, the occlusive implant 20 and/or the expandable framework 22 is shown in the deployed configuration while the at least one piece of expandable foam 160 is in the compressed configuration to improve clarity.

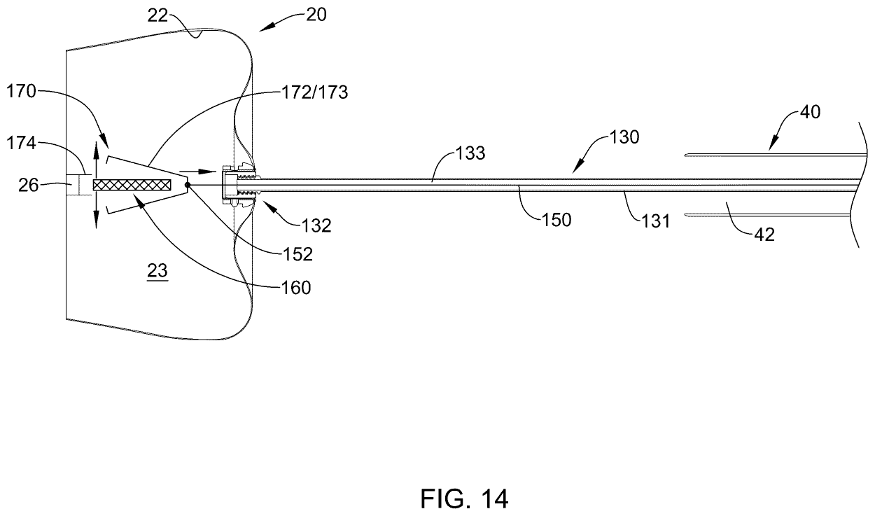

In some embodiments, proximal translation of the elongate strand 150 and/or tension applied to the elongate strand 150 may be configured to shift the retaining structure 170 from the retaining configuration to a release configuration, thereby releasing the at least one piece of expandable foam 160 within the interior 23 of the expandable framework 22, as seen in FIG. 14. While FIGS. 13-14 illustrate the at least one piece of expandable foam 160 as a single piece of expandable foam, this is merely exemplary, and it shall be understood that the at least one piece of expandable foam 160 may comprise a plurality of pieces of expandable foam as described herein.

In some embodiments, the retaining structure 170 may comprise a capsule 172. In some embodiments, the capsule 172 may comprise a plurality of elongate fingers 173. In some embodiments, the plurality of elongate fingers 173 may be fixedly secured to each other proximate a first end of the capsule 172 and/or the plurality of elongate fingers 173 and biased apart from each other towards an open configuration proximate a second end of the capsule 172 and/or the plurality of elongate fingers 173 opposite the first end of the capsule 172 and/or the plurality of elongate fingers 173. In some embodiments, the plurality of elongate fingers 173 may be self-biased apart from each other towards the open configuration proximate the second end of the capsule 172 and/or the plurality of elongate fingers 173. In some embodiments, the plurality of elongate fingers 173 may be spring biased apart from each other towards the open configuration proximate the second end of the capsule 172 and/or the plurality of elongate fingers 173. In some embodiments, the retaining structure 170 and/or the plurality of elongate fingers 173 may include a different biasing mechanism.

In some embodiments, the retaining structure 170 may comprise a coupling element 174 configured to engage with the capsule 172 and/or the plurality of elongate fingers 173. In some embodiments, the coupling element 174 may be configured to prevent the plurality of elongate fingers 173 from moving apart from each other proximate the second end of the capsule 172 and/or the plurality of elongate fingers 173 when the coupling element 174 is engaged with the capsule 172 and/or the plurality of elongate fingers 173. In some embodiments, the coupling element 174 and the capsule 172 and/or the plurality of elongate fingers 173 may be movable relative to each other to disengage the coupling element 174 from the capsule 172 and/or the plurality of elongate fingers 173. In at least some embodiments, proximal translation of the elongate strand 150 and/or tension applied to the elongate strand 150 may be configured to disengage the coupling element 174 and the capsule 172 and/or the plurality of elongate fingers 173 from each other, thereby permitting the plurality of elongate fingers 173 to shift toward the open configuration and release the at least one piece of expandable foam 160 within the interior 23 of the expandable framework 22.

In some embodiments, the coupling element 174 may be fixedly attached to the distal hub 26 and/or the expandable framework 22 and the capsule 172 and/or the plurality of elongate fingers 173 may be secured to, coupled to, and/or attached to the distal end 152 of the elongate strand 150, as seen in FIGS. 13-15. In some embodiments, the coupling element 174 may extend proximally from the distal hub 26 and/or the expandable framework 22. In some embodiments, the coupling element 174 may form and/or may be shaped like a cup or a partial cylinder open to one end. In some embodiments, the coupling element 174 may open proximally from the distal hub 26 and/or the expandable framework 22. In some embodiments, the coupling element 174 may be configured to receive at least a portion of the capsule 172 and/or the plurality of elongate fingers 173 therein. In some embodiments, the capsule 172 and/or the plurality of elongate fingers 173 may extend distally from the distal end 152 of the elongate strand 150 into the coupling element 174 in the retaining configuration such that the coupling element 174 prevents the plurality of elongate fingers 173 from shifting away from each other and/or towards the open configuration. The at least one piece of expandable foam 160 may be held in and/or by the retaining structure 170 during delivery and/or deployment of the occlusive implant 20. After shifting the occlusive implant 20 and/or the expandable framework 22 to the deployed configuration, the capsule 172 and/or the plurality of elongate fingers 173 may be disengaged from the coupling element 174 to release the at least one piece of expandable foam 160 within the interior 23 of the expandable framework 22, as seen in FIG. 14. In some embodiments, when the capsule 172 and/or the plurality of elongate fingers 173 is disengaged from the coupling element 174, the second end of the capsule 172 and/or the plurality of elongate fingers 173 may move radially outward and/or proximally as the plurality of elongate fingers 173 shifts away from each other and/or towards the open configuration.

In some embodiments, at least a portion of the retaining structure 170 may be configured to be removed from the interior 23 of the expandable framework 22 after releasing the at least one piece of expandable foam 160 within the interior of the expandable framework 22. In some embodiments, the capsule 172 and/or the plurality of elongate fingers 173 may be configured to be withdrawn proximally from the expandable framework 22 using the elongate strand 150, as seen in FIG. 15. In one example, the capsule 172 and/or the plurality of elongate fingers 173 may be configured to be withdrawn through the proximal hub 24 using the elongate strand 150. In some embodiments, the capsule 172 and/or the plurality of elongate fingers 173 may be configured to be withdrawn into and/or through the lumen 133 of the core wire 130 using the elongate strand 150. Other configurations are also contemplated.

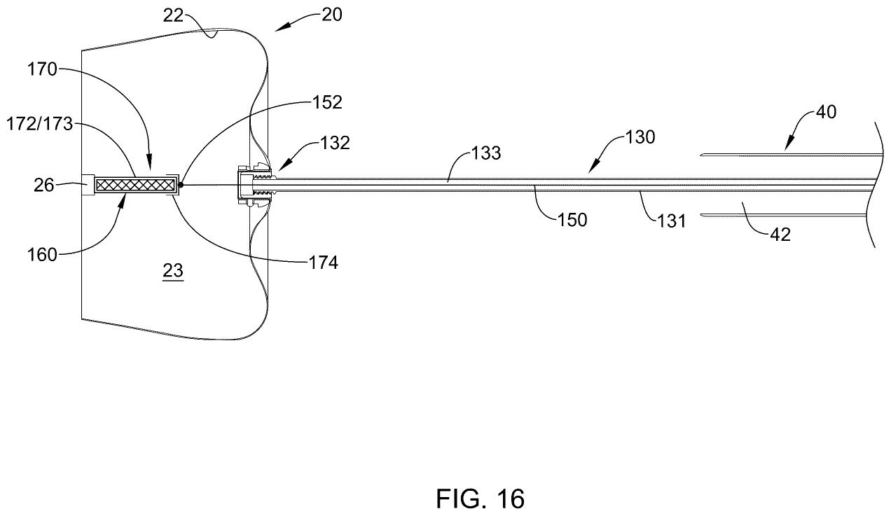

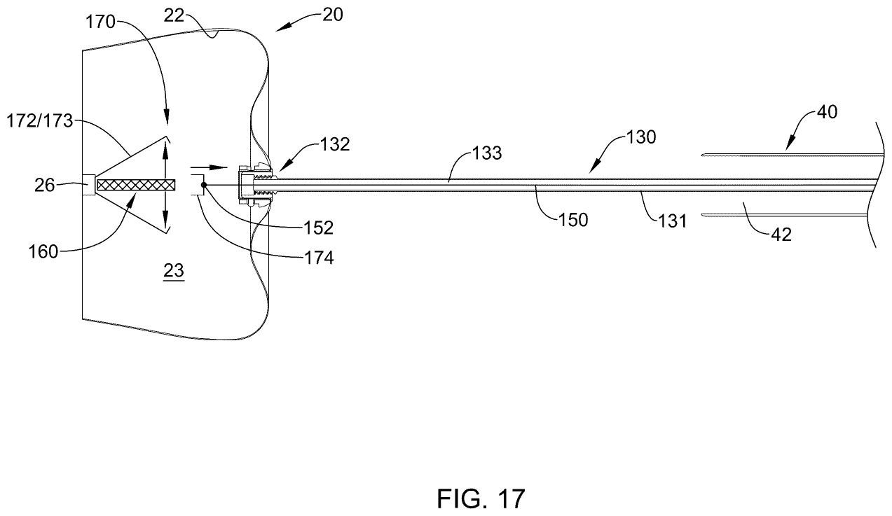

In some embodiments, the coupling element 174 may be secured to, coupled to, and/or attached to the distal end 152 of the elongate strand 150 and the capsule 172 and/or the plurality of elongate fingers 173 may be fixedly attached to the distal hub 26 and/or the expandable framework 22, as seen in FIGS. 16-18. In some embodiments, the coupling element 174 may extend distally from the distal end 152 of the elongate strand 150. In some embodiments, the coupling element 174 may form and/or may be shaped like a cup or a partial cylinder open to one end. In some embodiments, the coupling element 174 may open distally from the distal end 152 of the elongate strand 150. In some embodiments, the coupling element 174 may be configured to receive at least a portion of the capsule 172 and/or the plurality of elongate fingers 173 therein. In some embodiments, the capsule 172 and/or the plurality of elongate fingers 173 may extend proximally from the distal hub 26 and/or the expandable framework 22 into the coupling element 174 in the retaining configuration such that the coupling element 174 prevents the plurality of elongate fingers 173 from shifting away from each other and/or towards the open configuration. The at least one piece of expandable foam 160 may be held in and/or by the retaining structure 170 during delivery and/or deployment of the occlusive implant 20. After shifting the occlusive implant 20 and/or the expandable framework 22 to the deployed configuration, the capsule 172 and/or the plurality of elongate fingers 173 may be disengaged from the coupling element 174 to release the at least one piece of expandable foam 160 within the interior 23 of the expandable framework 22, as seen in FIG. 17. In some embodiments, when the coupling element 174 is disengaged from the capsule 172 and/or the plurality of elongate fingers 173, the second end of the capsule 172 and/or the plurality of elongate fingers 173 may move radially outward and/or distally as the plurality of elongate fingers 173 shifts away from each other and/or towards the open configuration.

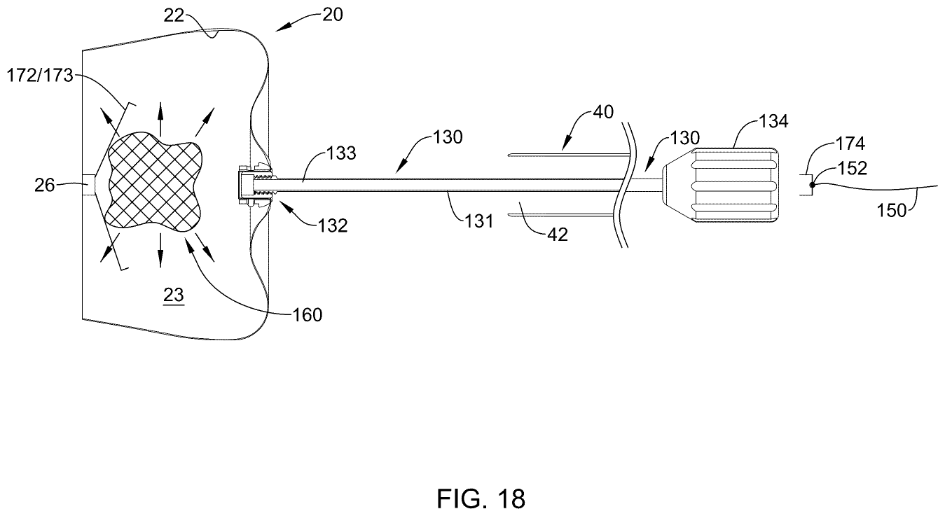

In some embodiments, at least a portion of the retaining structure 170 may be configured to be removed from the interior 23 of the expandable framework 22 after releasing the at least one piece of expandable foam 160 within the interior of the expandable framework 22. In some embodiments, the coupling element 174 may be configured to be withdrawn proximally from the expandable framework 22 using the elongate strand 150, as seen in FIG. 18. In one example, the coupling element 174 may be configured to be withdrawn through the proximal hub 24 using the elongate strand 150. In some embodiments, the coupling element 174 may be configured to be withdrawn into and/or through the lumen 133 of the core wire 130 using the elongate strand 150. Other configurations are also contemplated.

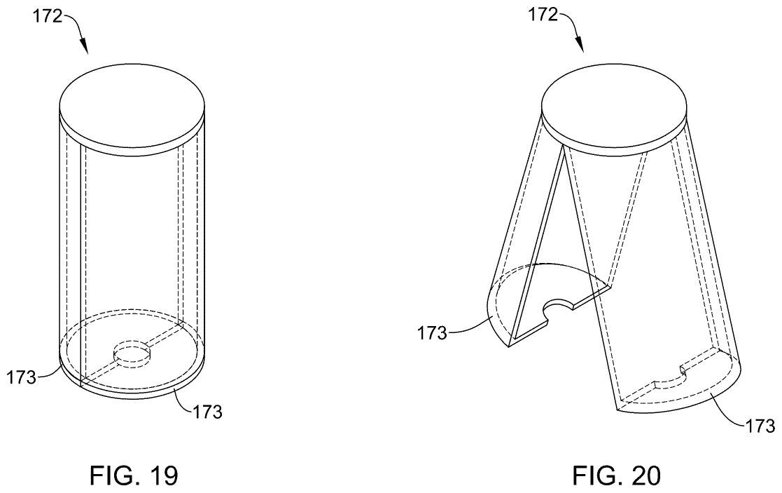

FIGS. 19-22 illustrate selected aspects of the capsule 172 and/or the plurality of elongate fingers 173. In some embodiments, the capsule 172 and/or the plurality of elongate fingers 173 may be formed from a tubular member or a cylindrical member. In some embodiments, the tubular member or the cylindrical member may be cut into the plurality of elongate fingers 173. In some embodiments, the tubular member may be a hypotube. In some embodiments, shapes and/or configurations for the tubular member other than circular cross-sectional shape may be used. In some embodiments, the tubular member may have a polygonal cross-sectional shape. Other configurations are also contemplated. As discussed herein, the plurality of elongate fingers 173 may be secured to each other proximate the first end of the capsule 172 and/or the plurality of elongate fingers 173 and may be biased apart from each other proximate the second end of the capsule 172 and/or the plurality of elongate fingers 173.

In some embodiments, the capsule 172 and/or the plurality of elongate fingers 173 may comprise two elongate fingers, as seen in FIGS. 19-20. In some embodiments, the capsule 172, the plurality of elongate fingers 173, and/or the two fingers may be formed by cutting the tubular member or the cylindrical member in half. In some embodiments, the capsule 172, the plurality of elongate fingers 173, and/or the two fingers may form a clamshell arrangement configured to shift between the retaining configuration, seen in FIG. 19, and the open configuration, seen in FIG. 20.

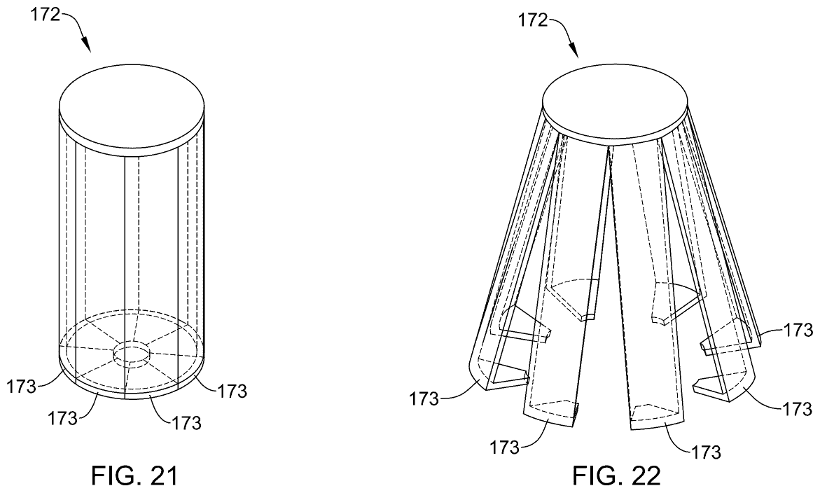

In some embodiments, the capsule 172 and/or the plurality of elongate fingers 173 may comprise more than two elongate fingers, as seen in FIGS. 21-22. In one example, the capsule 172 and/or the plurality of elongate fingers 173 may comprise eight elongate fingers. Other configurations, including but not limited to three elongate fingers, four elongate fingers, five elongate fingers, six elongate fingers, seven elongate fingers, nine elongate fingers, ten elongate fingers, etc., are also contemplated. In some embodiments, the capsule 172, the plurality of elongate fingers 173, and/or the more than two elongate fingers (e.g., the eight elongate fingers, etc.) may be formed by cutting the tubular member or the cylindrical member into a plurality of strips configured to shift between the retaining configuration, seen in FIG. 21, and the open configuration, seen in FIG. 22. In some embodiments, the capsule 172, the plurality of elongate fingers 173, and/or the more than two elongate fingers (e.g., the eight elongate fingers, etc.) may be formed similar to a collet. Other configurations and/or quantities of elongate fingers are also contemplated.

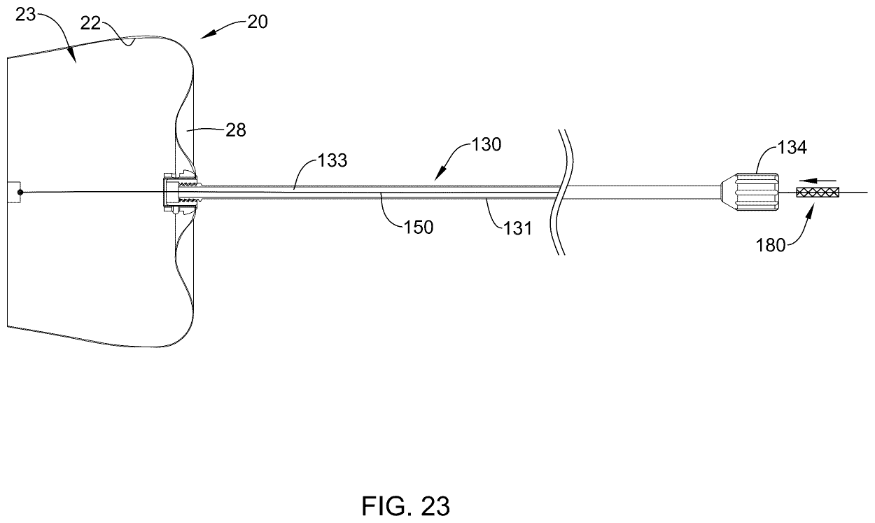

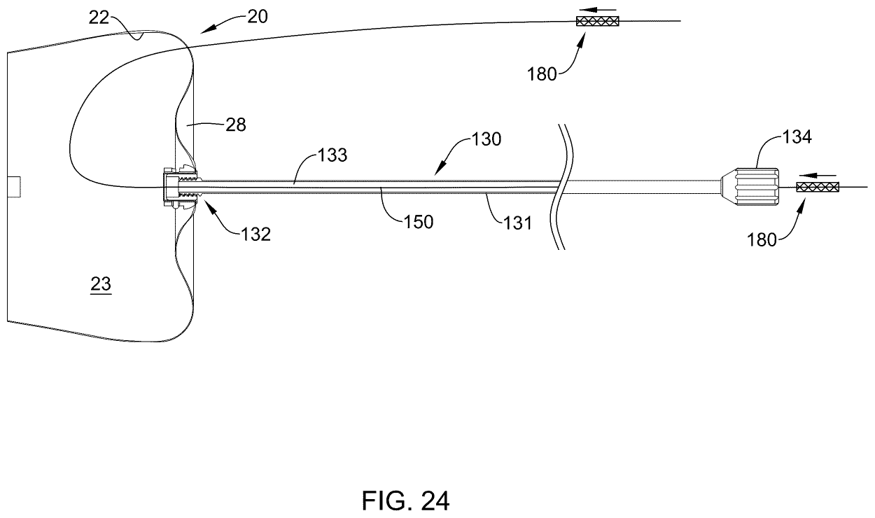

In some embodiments, the occlusive implant system 10 may comprise at least one drug-eluting capsule 180 configured to slide along and/or over the elongate strand 150 into the interior 23 of the expandable framework 22, as seen in FIGS. 23-25. In some embodiments, the at least one drug-eluting capsule 180 may comprise one drug-eluting capsule (e.g., FIG. 23), two drug-eluting capsules (e.g., FIG. 24), three drug-eluting capsules, etc. In some embodiments, the at least one drug-eluting capsule 180 may be used in addition to or alternatively to the at least one piece of expandable foam 160 described herein. The at least one drug-eluting capsule 180 may take any one of a variety of shapes and/or forms suitable for advancement along and/or over the elongate strand 150. For exemplary purposes only, the at least one drug-eluting capsule 180 is illustrated in FIGS. 23-25 schematically.

In some embodiments, each drug-eluting capsule of the at least one drug-eluting capsule 180 may be configured to slide along and/or over the first portion of the elongate strand 150. In some embodiments, each drug-eluting capsule of the at least one drug-eluting capsule 180 may be configured to slide along and/or over the second portion of the elongate strand 150. In some embodiments, each drug-eluting capsule of the at least one drug-eluting capsule 180 may be configured to slide along and/or over the different portions of the elongate strand 150 (e.g., one drug-eluting capsule over the first portion and one drug-eluting capsule over the second portion, one drug-eluting capsule over the first portion and two drug-eluting capsules over the second portion, etc.). Other configurations, including various combinations thereof, are also contemplated.

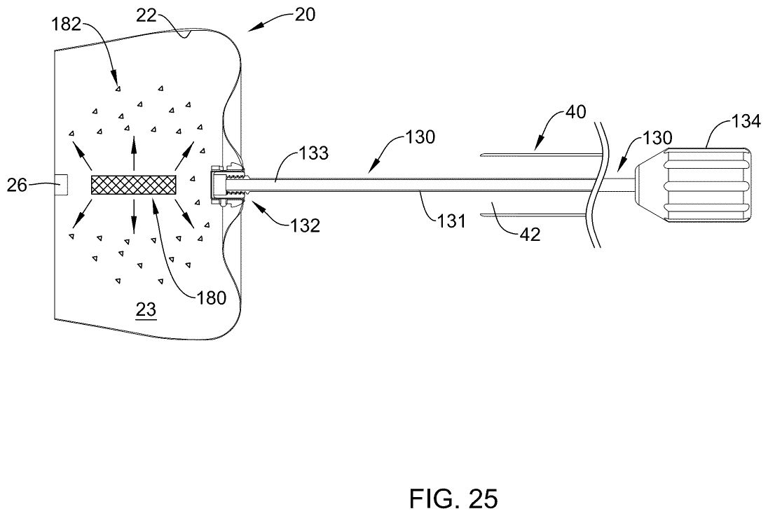

In some embodiments, the at least one drug-eluting capsule 180 may be configured to release a therapeutic agent 182 into the interior 23 of the expandable framework 22, as seen in FIG. 25. Some suitable but nonlimiting examples of therapeutic agents are described below. In some embodiments, the at least one drug-eluting capsule 180 may be configured to release a single therapeutic agent into the interior 23 of the expandable framework 22. In some embodiments, the at least one drug-eluting capsule 180 may be configured to release a plurality of therapeutic agents into the interior 23 of the expandable framework 22. In some embodiments, each drug-eluting capsule of the at least one drug-eluting capsule 180 may be configured to release a single therapeutic agent into the interior 23 of the expandable framework 22. In some embodiments, each drug-eluting capsule of the at least one drug-eluting capsule 180 may be configured to release a different therapeutic agent into the interior 23 of the expandable framework 22 (e.g., each one of three drug-eluting capsules may be configured to release one of three different therapeutic agents, etc.).

In some embodiments, the at least one drug-eluting capsule 180 may be configured to release the therapeutic agent 182 over time. In some embodiments, the at least one drug-eluting capsule 180 may be configured to release the therapeutic agent 182 after a predetermined amount of time. In some embodiments, the at least one drug-eluting capsule 180 may be configured to release the therapeutic agent 182 after a predetermined amount of time that the at least one drug-eluting capsule 180 has been disposed within the interior 23 of the expandable framework 22 and/or within the target site (e.g., the left atrial appendage). In some embodiments, each drug-eluting capsule of the at least one drug-eluting capsule 180 may be configured to release the therapeutic agent 182 disposed therein after a predetermined amount of time. In some embodiments, each drug-eluting capsule of the at least one drug-eluting capsule 180 may be configured to release the therapeutic agent 182 disposed therein after a different predetermined amount of time (e.g., each drug-eluting capsule may be configured to release the therapeutic agent 182 disposed therein at different times, etc.). Other configurations are also contemplated.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the occlusive implant, the delivery sheath, the core wire, the expandable framework, the occlusive element, the capsule, the elongate fingers, the elongate strand, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An occlusive implant system, comprising:
a core wire having a lumen extending therethrough;
an occlusive implant releasably coupled to a distal end of the core wire, the occlusive implant comprising an expandable framework configured to shift between a collapsed configuration and a deployed configuration; and
an elongate strand extending along the core wire to the expandable framework;
wherein the elongate strand extends into an interior of the expandable framework in the deployed configuration.

2. The occlusive implant system of claim 1, wherein the elongate strand extends within the lumen of the core wire to the expandable framework.

3. The occlusive implant system of claim 1, wherein a distal end of the elongate strand is secured to the expandable framework.

4. The occlusive implant system of claim 3, wherein the distal end of the elongate strand is secured to a proximal portion of the expandable framework.

5. The occlusive implant system of claim 3, wherein the distal end of the elongate strand is secured to a distal hub of the expandable framework.

6. The occlusive implant system of claim 3, wherein the elongate strand is configured to remain coupled to the expandable framework after the core wire has been decoupled from the expandable framework.

7. The occlusive implant system of claim 1, further comprising an occlusive covering disposed on a proximal portion of the expandable framework.

8. The occlusive implant system of claim 1, further comprising at least one piece of expandable foam configured to slide over the elongate strand into the interior of the expandable framework, the at least one piece of expandable foam being configured to remain within the interior of the expandable framework permanently.

9. An occlusive implant system, comprising:
a core wire having a lumen extending therethrough;
an occlusive implant releasably coupled to a distal end of the core wire, the occlusive implant comprising an expandable framework configured to shift between a collapsed configuration and a deployed configuration;
an elongate strand extending along the core wire to the expandable framework, wherein the elongate strand extends into an interior of the expandable framework in the deployed configuration; and
at least one piece of expandable foam configured to slide in a compressed configuration along the elongate strand into the interior of the expandable framework, wherein the at least one piece of expandable foam is configured to shift to an expanded configuration within the interior of the expandable framework.

10. The occlusive implant system of claim 9, wherein in the expanded configuration the at least one piece of expandable foam is configured to fill at least 50% of the interior of the expandable framework in the deployed configuration.

11. The occlusive implant system of claim 9, wherein a first portion of the elongate strand extends within the lumen of the core wire to the expandable framework.

12. The occlusive implant system of claim 11, wherein a second portion of the elongate strand extends alongside and external to the core wire to the expandable framework.

13. The occlusive implant system of claim 12, wherein a first piece of the at least one piece of expandable foam is configured to slide along the first portion of the elongate strand into the interior of the expandable framework and a second piece of the at least one piece of expandable foam is configured to slide along the second portion of the elongate strand into the interior of the expandable framework.

14. The occlusive implant system of claim 9, wherein the at least one piece of expandable foam is disposed outside of the expandable framework prior to shifting the expandable framework to the deployed configuration.

15. An occlusive implant system, comprising:
a core wire having a lumen extending therethrough;
an occlusive implant releasably coupled to a distal end of the core wire, the occlusive implant comprising an expandable framework configured to shift between a collapsed configuration and a deployed configuration;
at least one piece of expandable foam disposed in a compressed configuration within an interior of the expandable framework in the collapsed configuration;
a retaining structure holding the at least one piece of expandable foam in the compressed configuration, wherein the retaining structure is engaged with the expandable framework in a retaining configuration; and
an elongate strand extending along the core wire to the expandable framework, wherein the elongate strand extends into the interior of the expandable framework and is coupled to the retaining structure.

16. The occlusive implant system of claim 15, wherein proximal translation of the elongate strand is configured to shift the retaining structure from the retaining configuration to a release configuration, thereby releasing the at least one piece of expandable foam within the interior of the expandable framework.

17. The occlusive implant system of claim 16, wherein at least a portion of the retaining structure is configured to be removed from the interior of the expandable framework after releasing the at least one piece of expandable foam within the interior of the expandable framework.

18. The occlusive implant system of claim 15, wherein the retaining structure comprises a plurality of elongate fingers fixedly secured to each other proximate a first end of the plurality of elongate fingers and biased apart from each other proximate a second end of the plurality of elongate fingers opposite the first end.

19. The occlusive implant system of claim 18, wherein the retaining structure comprises a coupling element configured to prevent the plurality of elongate fingers from moving apart from each other proximate the second end of the plurality of elongate fingers.

20. The occlusive implant system of claim 19, wherein proximal translation of the elongate strand is configured to disengage the coupling element and the plurality of elongate fingers from each other.
